(19) United States
(12) Patent Application Publication
(10) Pub. No.: US 2021/0145615 A1
(43) Pub. Date: May 20, 2021

(54) AN IMPROVED CORONARY STENT DELIVERY SYSTEM FOR BIFURCATION LESIONS

(71) Applicant: Alok RANJAN, Surat (IN)

(72) Inventor: Alok RANJAN, Surat (IN)

(21) Appl. No.: 16/623,222

(22) PCT Filed: Jun. 15, 2018
(86) PCT No.: PCT/IN2018/050395

§ 371 Date: Dec. 16, 2019

(51) Int. Cl. A61F 2/856; A61F 2/958; A61F 2/954

(52) U.S. Cl. CPC A61F 2/856; A61F 2002/821; A61F 2/954; A61F 2/958

(57) ABSTRACT

The invention relates to an improved coronary stent delivery system to address bifurcation lesion, a modification (channel) in stent 5 delivery system. The said system comprises steps a) to accommodate an extra PTCA wire between the stent and the underlying balloon with first wire in the main branch (MB) port and the side branch wire in channel provided for it in between stent and underlying balloon; b) load the stent on the MB wire and also pass 10 the SB wire in the channel provided for it so that once stent is inflated in the main branch and the balloon is removed after inflation, the side branch is accessible as there is SB wire is already inside the stent; c) another balloon or stent to be used to open the side branch in case there is serious compromise in flow in 15 side branch or if 2 stents strategy is planned.

### FIELD OF THE INVENTION

Present invention relates to an improved system for coronary stent. More particularly, the present invention relates to a stent delivery platform and an improved coronary stent and a stent delivery platform having improvement in the stent design to deal with bifurcation lesion.

### PRIOR ART

A coronary stent is a tube-shaped device placed in the coronary arteries that supply blood to the heart, to keep the arteries open in the treatment of coronary heart disease. They are usually made of metal mesh.

Following is the procedure for coronary stenting:
1. The stent, which is mounted on a balloon at the tip of the catheter, is guided through the artery to the blockage.
2. At the blockage, the balloon is inflated and the stent expands and locks into place inside the artery.
3. The stent stays in the artery permanently to hold it open and improve blood flow to the heart.
4. Once the stent is in place, the balloon catheter is removed.

Coronary angioplasty and stenting is a common procedure to treat blockages in coronary (heart) arteries. Stents used in coronary arteries are usually balloon expandable. The stent is mounted over a balloon and once it is placed across a blockage (lesion) the balloon is inflated and the stent expands and conforms to the vessel wall. Size and length of the stent depends on the lesion characteristics. It is available in different length and diameter to deal with all sizes of arteries. Any artery in the body can have branches and so can the coronary arteries. If the lesion in the artery has some branches coming out from that segment then stent may block the flow in that branch. This happens as the stent struts can cover the opening of that branch. If the side branch is large it can lead to adverse outcome in some cases. Main aim is to treat the main branch and to keep the side branch open as far as possible. Such lesion where a branch is involved is called as bifurcation (involving 2 branches) lesions. There could also be trifurcation or quadrifurcation lesions also but the incidences of those lesions are less. Bifurcation lesions constitute nearly about 20% of all coronary lesions.

Bifurcation lesions are considered as complex lesions to treat due to poor outcome as compared to lesions where large side branches are not involved. One approach is to use conventional stent and if the flow to side branch is jeopardised then open the side branch by doing balloon dilatation (angioplasty) or stenting. Passing an angioplasty wire in the side branch through struts of main branch stent is required for this part of the procedure. After passage of wire, it is dilated with balloon and stented if required. Many strategies are there to do such procedure. The basic steps are to wire both the branches to start with. The second step is to dilate and stent one of the branches (mostly the main branch) and then optimising result on that branch by use of additional balloon in proximal part of the main branch stent. This is required as the arteries gradually taper off to smaller diameter as they go down. After this if the side branch flow is not good then the wires are interchanged as the side branch wire is beneath or outside the first stent. This is the most crucial step as it can be challenging to recross through the stent struts of main branch. Crossing through the distal most strut is shown to have better results. Once wires are replaced, either balloon dilatation and/or stenting can be performed in the side branch to improve the result. Final step is to do simultaneous balloon inflation (‘Kisiing balloon technique’) in 2 branches to minimise stent distortion.

These steps are complex and depend on the operator experience and his judgement since only one design of stent is available, which is an inherent limitation of the coronary stent. The conventional coronary stent is shown for ready reference. New dedicated bifurcation stents for bifurcation are being developed and tested in trials worldwide to address bifurcation lesion in a more effective manner. But they are not widely available.

Conventional Bifurcation Stenting: Limitations
1. Maintaining access to side branch (SB) throughout the procedure.
2. Main branch (MB) stent struts jailing SB resulting in difficulty in rewiring SB or passing balloon/stent into SB through the stent struts.
3. Distortion of MB stent by SB dilatation; inability to fully cover and scaffold ostium of SB.
4. Inability of stent structure to withstand SB balloon dilatation and deformation.
5. Operator skills and technical experience.

Dedicated Bifurcation Stents: Limitations
1. Double balloon stent delivery system is bulkier (>6Fr catheters), limits use in calcified and tortuous lesion and wire wrap is common.
2. Stents with preformed SB aperture requires accurate positioning with little tolerance for incorrect placement.
3. SB specific stent commit the operator to stent both branches.
4. Not all dedicated bifurcation stent provide complete coverage of SB ostium (e.g., Frontier, Stentys SDS).
5. Not widely available.

### OBJECT OF THE PRESENT INVENTION

Object of the present invention aims at developing an improved coronary stent and a stent delivery platform.

It is also the object of the present invention is to create a channel between balloon and stent for side branch wire (port for the main branch or the parent branch remains as it is).

Yet another object of the present invention is to modify the stent design in such a way that the distortion of the main branch stent struts is minimized thereby helping to reduce blockage in side branch (SB) and also reduce the chance of stent thrombosis (blood clot in the stent).

Yet another object of the present invention is to develop a dedicated side branch stent to deal with the challenges of side branch lesion in a more effective manner.

It is also the object of the present invention is to eliminate the short comings and drawbacks of the prior art.

### STATEMENT OF THE PRESENT INVENTION

The invention provides an improved coronary stent delivery system to address bifurcation lesion by a modification (channel) in stent delivery system comprises step:
a. to accommodate an extra PTCA wire between the stent and the underlying balloon (with the port for main branch [MB] wire remain unchanged) for the side branch (SB);
b. to load the stent on the MB wire and also pass the SB wire in the channel provided for it so that once stent is inflated (and the balloon is removed after inflation) in the main branch, the side branch is accessible as there is SB wire inside the stent;
c. another balloon or stent to be used to open the side branch in case there is serious compromise in flow in side branch or if 2 stents strategy is planned.

### BRIEF DESCRIPTION OF THE DRAWINGS

FIG. 1 is a coronary stent and delivery platform as per the prior art or conventional method;

FIG. 2 is a coronary stent and delivery platform as per the prior art showing the location marker with inflated balloon with drug coated stent;

FIG. 3 is an improved stent design according to the present invention;

FIG. 4 is an improved stent design according to the present invention with multiple channels for side branch wire. Only one channel will be used in one patient but presence of multiple channels will help to treat more patients as the lesion characteristics are not identical in patients;

FIG. 5 shows MB (main branch) wire in main or bigger branch and SB (side branch) wire in the side or smaller branch;

FIG. 6 shows stent in main branch or branch with difficult angle along with MB wire in the main port of the standard stent delivery system; the SB wire is outside the stent; between artery and stent in MB

FIG. 7 shows Short balloon (yellow color) in proximal part of stent to resize it according to diameter of proximal part of stent; Here also SB wire is outside the stent. It is recommended to keep SB wire outside the MB stent till this stage in conventional technique

FIG. 8 shows Steps of rewiring the branches: the MB wire (blue color) is now placed in side branch through the MB stent struts keeping SB wire as road map;

FIG. 9 shows some ‘distortion of MB stent struts at the orifice of side branch as the site where the wire is re-crossing into the side branch through the stent struts, it will vary in patient to patient opening up of MB stent struts at SB orifice is shown as ‘gap’ in stent outline across side branch orifice);

FIG. 10 shows Channel on the Balloon to accommodate SB wire;

FIG. 11 shows Channel within stent struts to accommodate SB wire;

FIG. 12 shows The struts covering the side branch origin can be modified to facilitate crossing of PTCA balloon or stent and site can be modified to achieve either complete ostial coverage OR coverage of non carinal segment of side branch in the proposed change of design;

FIG. 13 shows complete osteal coverage of side branch in the proposed change of design;

FIG. 14A showing ‘degree’ of stent protrusion in main branch depending on the angle between main branch and side branch;

FIG. 14B shows in order to avoid protrusion of stent into main branch a bevelled stent is proposed. Bevelled stent in side branch will minimise the stent protrusion into main branch;

FIG. 15A shows Bevelled stent (blue color) shown with the underlying balloon (outline by yellow color). Main branch (MB) and side branch (SB) wires are also shown to understand deployment of this type of stent;

FIG. 15B shows Bevelled stent (Blue color) as positioned in side branch. The bevelled edge helps to minimise stent protrusion in main branch;

FIG. 16 shows another Dedicated SB Stent design to address side branch issues; Stent with long ‘overhanging’ balloon on proximal part of stent

### DESCRIPTION OF THE INVENTION

Referring to FIG. 1: Explains the stenting procedure according to prior art in an artery. The first step is to pass a very thin PTCA (Percutaneous transluminal coronary angioplasty) wire across the lesion. Over this wire a stent system (stent mounted over a balloon) is placed across the lesion under fluoroscopic (X-ray) guidance. Once the stent is placed in optimal position, the balloon is inflated to expand the stent. After satisfactory expansion of stent, the balloon is deflated and the PTCA wire along with balloon is removed, leaving behind the stent inside the artery. In this figure the lesion is present in a part of artery where there is no branching. However, if there are other side branches coming out from the stenosed (narrowed) segment of artery then the flow in the side branch may be compromised by stent placement. In such situations, bifurcation stenting/intervention is planned if the side branch is of good caliber and supplies substantial area of myocardium (heart muscle). These bifurcation stenting procedure are more complex than the usual stenting procedure.

Referring to FIG. 2: Magnified view of stent system of prior art in inflated state: Stent is mounted (comes in already mounted condition in present generation of stents) on the balloon on delivery system. There are radio opaque location markers to assist in appropriate placement of stent across the lesion. The location markers are just outside the stent margins to help placement of stent in a manner that it covers the entire length of lesion and the edges are placed in normal segment of artery to minimize complications. Stent system comes in mounted state with deflated underlying balloon and it (balloon) is inflated only when it is in proper position. The balloon is deflated again and removed from the artery to complete the procedure.

Referring to FIG. 3: according to invention proposed changes in stent delivery system to address bifurcation lesion: Once stent (Blue line) is inflated over the balloon (yellow line) it may compromise the flow in side branch. In order to avoid it, the new system is planned. There should be a modification (channel) in stent delivery system so that it can accommodate an extra PTCA wire between the stent and the underlying balloon. So the procedure will be as follows; first wire the main branch (MB) and the side branch (SB). Load the stent on the MB wire and also pass (backload as for MB) the SB wire in the channel provided for it. Because of this modification once stent is inflated (and the balloon is removed after inflation) in the main branch, the side branch is accessible as there is SB wire inside the stent. Another balloon or stent can be used to open the side branch in case there is serious compromise in flow in side branch. Placement of MB stent is to be done in such a manner that the SB wire crosses through distal most strut of stent to get perfect result.

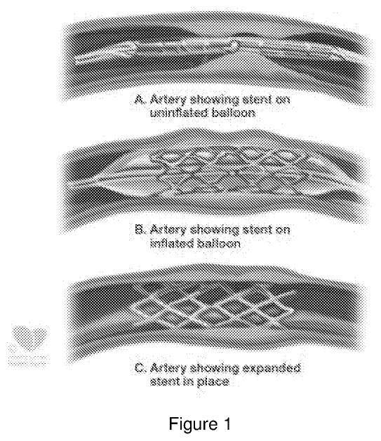

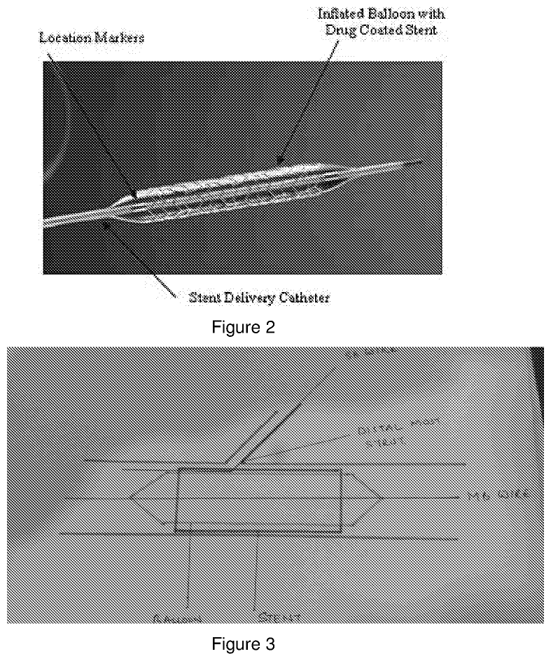

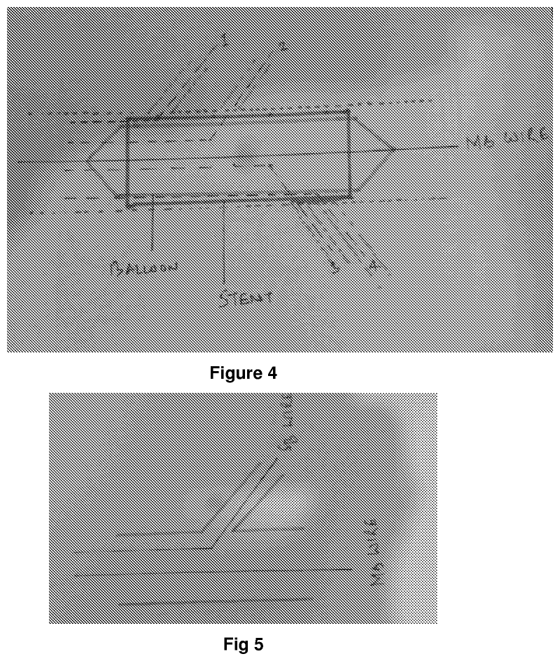

Referring to FIG. 4: Basic structure is same as the FIG. 3. In FIG. 3, only one site of side branch origin is shown. However, in real world situations, side branch origin is not fixed and it may arise at any site and its origin may vary along the lesion length. One fixed stent delivery system will not work for every situation. If channels are placed at different sites so that depending on the site of origin of side branch the appropriate channel can be used. So if there is provision of multiple channels or one channel but if it can be used at different sites (as per individual case) the stent delivery system's utility can be increased to most cases of bifurcation stenting. Pink broken lines depict the possibility of side branch origin from 4 different sites from the main branch. Blue broken lines (1, 2, 3 and 4) inside the pink broken lines denotes PTCA wire in such case.

Only one side branch of greatest significance will be addressed but it can arise from anywhere from the main branch (green broken lines).

The new improved stent and stent delivery platform according to the present invention and as shown in FIGS. 3 and 4 facilitate to overcome the difficulties associated with the prior art and also simplifies the procedure. According to the present invention, creating a channel between the balloon and stent for the side branch wire will help to simplify bifurcation stenting strategies. A central wire is always there in all stent delivery system (SDS) for deployment of the stent. According to the present invention, the additional channel can be provided on the balloon surface itself or on the stent so that after the stent deployment in the main branch the side branch wire is within the stent and not outside of it. Because of this provision, there is no need to recross the stent struts to address side branch lesion. This embodiment will save time, and any new operator will perform the procedure even without any experience of bifurcation lesions.

According to another embodiment of the present invention multiple channels are present. Any one of them can be used as the anatomy of the bifurcation lesion is not identical in patients so that it can be used in most cases. The present dedicated stents according to the prior art are not useful in many cases as proper exit sites are not available on them, which is an inherent limitation of the dedicated bifurcation stent. The fixed relation between stent and the side branch exit site in a dedicated bifurcation stent system prevents its use in most cases as there is no fixed relation between branches in patients. So multiple channels in the stent according to the present invention will increase its utility even though only one channel will be used in one given case.

According to the present invention, Stent design around the side branch wire can be modified in such a way so that the distortion of main branch stent struts is minimised and this will help to reduce reblockage in side branch and also reduce the chances of stent thrombosis (blood clot in stent). This will improve the long term results of bifurcation lesions. Due to limitation of the conventional design as shown in FIGS. 1 and 2 the present poor outcome of bifurcation stenting is related to side branch compromise after conventional stents and techniques used to deloy the same. According to another embodiment of the present invention, 2 dedicated side branch stents are proposed which deals with the challenges of side branch lesion in a more effective manner.

The Details of the Stent Delivery System:—

Bifurcation technique with new stent design steps:—
Step I: wire both branches
Step II: deploy MB stent
Step IIIa: finish with KBT (kissing Balloon Technique) or POT (Proximal Optimisation Technique) if side branch result is satisfactory
Step IIIb: stent the SB branch and finish with KBT (if stent deployment is planned)
Step I: Wiring both branches
step I remains same for the old and the proposed change in stent design
basic step.

Referring to FIG. 5, MB (main branch) wire in main or bigger branch and SB (side branch) wire in the side or smaller branch. Artery is outlined by green color and wires with blue color.

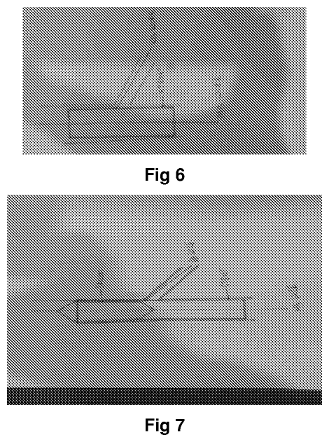

Step II: Conventional technique: stenting the first branch
usually the main branch or branch with more difficult angle
stent as per ‘distal’ reference vessel diameter
   Referring to FIG. 6 comprises:

Stent in main branch or branch with difficult angle along with MB wire in the main port of the standard stent delivery system. Note that the stent may not be adequately covering the proximal part of artery as stent is selected as per distal diameter of artery. However it is optimised with a bigger PTCA balloon (called POT technique; step III). Also note that now the SB wire is in between the artery and the stent in main branch in conventional technique at this step.

New proposed design: Step II
stent is selected as in the conventional technique. However, the SB wire is backloaded (as wires are already in place) in the ‘channel’ provided/proposed and the MB wire remains in the main port of new system.

Once the stent is deployed, the SB wire is now inside the stent and not outside as in the conventional system.

Referring to FIG. 3, MB wire (blue color) in the main port and SB wire (blue color) in the ‘channel’ between stent and the underlying balloon (yellow color). So after inflation the SB wire is inside and not outside the stent (blue line outside balloon). The placement of stent can be done in such a manner that the SB wire is in the distal most strut of the part of stent strut covering the orifice of side branch (as recommended in present techniques). The POT technique may be used to optimise size in the proximal part of stent if necessary as in the conventional technique.
Step III: POT (proximal optimisation technique)
if required, this step is same in both the stent designs. However, it may not be required as an additional step with the new design if the final kissing balloon achieves good result in proximal part of main branch stent.
however, there may be some other advantages in the new stent design as for POT, a definite length of stent is required in the proximal part of stent to accommodate the shortest balloon; it may be challenging or unnecessary in some conditions. as POT is not essential in new stent design these uncommon challenges may still be easier with this design
   Referring to FIG. 7, Short balloon (yellow color) in proximal part of stent to resize it according to diameter of proximal part of stent. This step is essential in existing stent design to facilitate rewiring of arteries (step IV).
   However in new design, POT can be done if kissing balloon technique (KBT) is not planned or it can be skipped and KBT can be performed to finish the case or 2 stents strategy can be planned
Step IV: rewiring both the branches
required only in the existing design
most crucial step if 2 stents or kissing balloon technique is planned
side branch wire should be in the ‘distal’ most strut (proximal if ‘crush technique [A type of 2 stent strategy in bifurcation lesions] is planned)
   It is challenging to pass through distal most strut in ‘real’ world setting and also due to two dimensional view in three dimensional setting
Pre dilatation of side branch may require more than 1 PTCA balloon.

Referring to FIG. 8, Steps of rewiring the branches: the MB wire (blue color) is now placed in side branch keeping SB wire as road map (fig b). After placing MB wire into side branch, the SB wire is now taken out and placed in the main branch (FIG. 8A). This ensures that now both the wires are inside the stent and further procedure on the side branch can be performed without significant deforming the already deployed stent. This is ‘crucial’ step in the existing stent delivery system. This step is not required in the new design as both wires are already inside the stent

Step V/VI: 2^{nd} branch stent or KBT
aims:
   to keep ‘carina’ as clean as possible
   to minimise ‘metal’ as far as possible around side branch orifice.

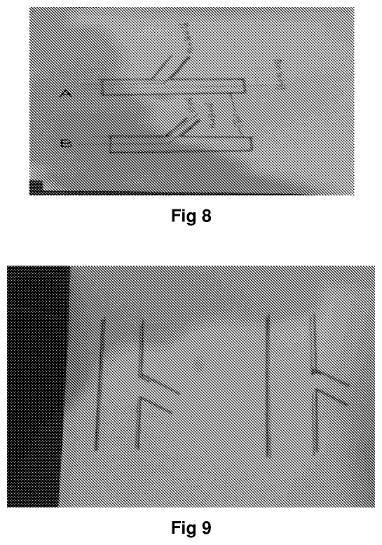

Referring to FIG. 9, some ‘distortion of stent struts at the orifice of side branch, as it is the site where the wire is recrossing into the side branch through the stent struts and site of crossing will vary in patient to patient (opening of MB stent struts is shown as ‘gap’ in stent outline across side branch orifice). This distortion can be minimised with new design

Step V/VI: Advantages with new stent design
carina as well as side opposite to carina can be as clean as possible.
minimal ‘heap up’ of metal in new stent design
channel for SB wire between stent and underlying balloon
   provision for multiple channels
SB dilatation to be facilitated with change in strut design around the entry point of ‘channel’
SB ostium coverage to be facilitated with change in strut design around the entry point of ‘channel.
   Referring to FIG. 4 multiple ‘channels’ if possible: site of multiple ‘channels’ are shown as broken blue wires no. 1, 2, 3 and 4. Such channels will make the new design easier to be used in all kinds of bifurcation lesions (as branches may come out at different positions on the main branch in ‘real’ world situations); a major limiting factor in the ‘dedicated’ bifurcation stent delivery system

Channels for SB Wire
on the balloon
   change in balloon material
   metallic base on the stent

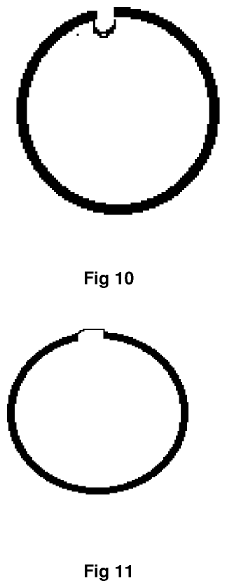

Referring to FIG. 10, for SB wire
should not damage the underlying balloon
   metal base
multiple channels to make the platform useful in almost all bifurcations
multiple channels can be at 180°, 120° or 90°

Channel within Stent Struts
Referring to 11, after deployment the struts should be well expanded
wire inside the stent so that after deployment it remains inside the stent

2Nd Change in Stent Design
the strut through which the SB wire is passing (around the entry point of ‘channel’), can be designed in such a way that it accommodates the side branch (SB) size balloon
due to this adjustment, no other PTCA balloons may be needed for predilatation of side branch.

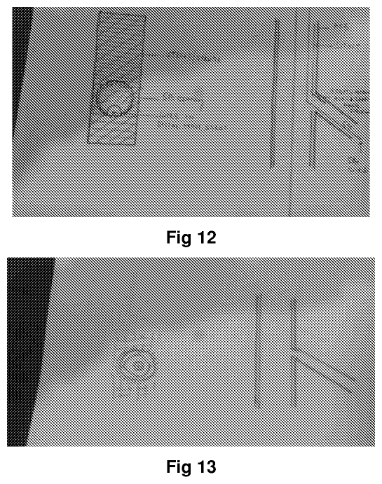

3Rd Change in Stent Design
Minimise ‘sliding/folding door’ approach
Minimise ‘heap’ of metal on wall opposite to carina
This will decrease chance of SB restenosis as well as thrombosis
Changes in struts design so as to properly scaffold the SB ostium
Minimise ‘Sliding/Folding’ of struts.
Referring to FIG. 12, Further minor changes in new stent design: The struts covering the side branch origin can be modified to facilitate crossing of PTCA balloon or stent and site can be modified to achieve either complete ostial coverage OR coverage of non carinal segment of side branch. In this figure it is shown to have non carinal coverage

Adequate Scaffolding of SB Ostium
In the FIG. 13 it is shown to have complete osteal coverage of side branch.

Advantages of New Stent Design
1. Operator friendly: Only skill needed is to wire both branches at the start of the procedure
2. Less procedure time/Radiation/Cost/Contrast
3. Wider applicability
4. Increased use of this technique will lead to more SB preservation; better long term outcome of disease
5. Strut design change around SB opening may further decrease incidence of restenosis or thrombosis
6. No need to change other existing hardware.

Limitations of New Stent Design
Increased chance of wire wrap (twisting)
? Increased chance of damage to the balloon on which stent is mounted
   PTCA wire for SB can be modified to minimise trauma to balloon
? Increased chance of stent dislodgement
? Increased chance of strut fracture/damage

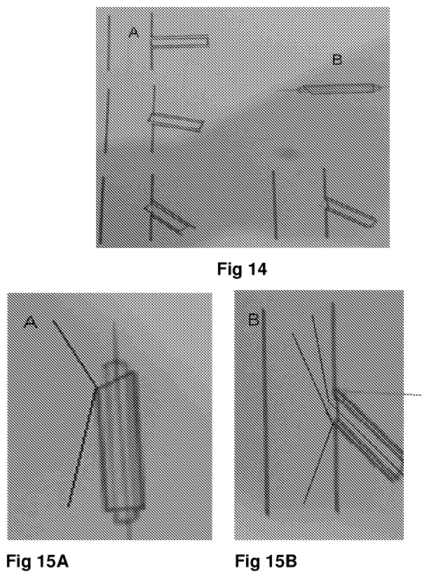

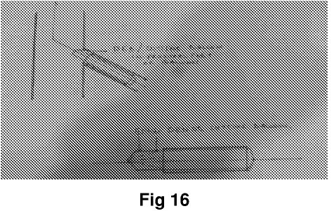

Dedicated SB Stents
Bevelled Stent
Based on the bifurcation angle
Slot for MB wire opposite to longer length of stent to guide proper position of stent in SB. This slot will help to position the stent at SB ostium
Referring to FIG. 14 A showing ‘degree’ of stent protrusion in main branch depending on the angle between main branch and side branch.
   To avoid protrusion of stent into main branch a bevelled stent is proposed.
   Bevelled stent in side branch will minimise the stent protrusion into main branch (FIG. 14 B).
Referring to FIG. 15 A: Bevelled stent (blue color) shown with the underlying balloon (outline by yellow color). Main branch (MB) and side branch (SB) wires are also shown to understand deployment of this type of stent.
FIG. 15 B: Bevelled stent (Blue color) as positioned in side branch. The bevelled edge helps to minimise stent protrusion in main branch.

2^{Nd} Design of Sb Stent
Conventional stent with longer overhanging length of balloon on the proximal end of the stent
This part of overhanging balloon can be drug eluting or have cutting balloon design
No need to protrude this stent into MB; the proximal overhanging part of balloon (drug eluting or cutting design) will help to minimise restenosis at SB ostium.
Less ‘metal’ at bifurcation site will help to minimise thrombosis

Dedicated SB Stent
Referring to 16, to avoid protrusion of stent in main branch, apart from bevelled stent design, another design can be planned. The underlying balloon overhangs the stent in its proximal part. After positioning this stent does not cover the ostium of side branch completely but the overhanging drug eluting or cutting balloon will help to dilate ostium, avoid stent protrusion and reduce ostium related restenosis.

Conclusions:
MB stent with channel for SB may be a game changer provided it is feasible to make such modifications
Dedicated SB stents are simple and may help to improve outcomes in 2 stent strategy
Tri or quadrifurcation lesion strategy may not change with the proposed design

### ADVANTAGES OF THE PRESENT INVENTION

1. Operator friendly: Only skill needed is to wire both branches at the start of the procedure
2. Less procedure time/Radiation/Cost/Contrast
3. Wider applicability
4. Increased use of this technique will lead to more SB preservation; better long term outcome of disease
5. No need to change existing hardware

1. An improved coronary stent delivery system to address bifurcation lesion, a modification (channel) in stent delivery system, comprises steps:
a. to accommodate an extra PTCA wires between the stent and the underlying balloon with first wire in the main branch (MB) port and the side branch wire in the ‘channel’ for SB wire;
b. load the stent on the MB wire (as per existing technique) and also pass the SB wire in the channel provided for it so that once stent is inflated and the balloon is removed after inflation in the main branch, so that the side branch is easily accessible as SB wire is already inside the stent (No need to recross);
c. another balloon or stent can be used to open the side branch in case there is serious compromise in flow in side branch or if 2 stent strategy is planned.

2. The stent delivery system as claimed in claim 1 wherein the placement of MB stent is to be done in such a manner that the SB wire passes through the distal most strut of stent to get perfect result.

3. An improved coronary stent delivery system to address bifurcation lesion, a modification (channel) in stent delivery system, comprises:
a. Step 1—Wire both branches;
b. Step 2—Deploy MB stent;
c. Step 3— Finish with KBT or POT if side branch result is satisfactory;
d. Step 4—Stent the SB branch and finish with KBT (if 2 stents deployment is planned).

4. The stent delivery system as claimed in claim 1 wherein the said Stent selected as in the conventional technique, the SB wire is backloaded (as wires are already in place) in the ‘channel’ provided/proposed and the MB wire remains in the main port of new system such that once the stent is deployed, the SB wire is now inside the stent and not outside as in the conventional system.

5. The stent delivery system as claimed in claim 1 wherein MB wire (blue) in the main port and SB wire (blue) in the ‘channel’ between stent and the underlying balloon (yellow color), so that after inflation the SB wire is inside and not outside the stent (Blue line outside balloon) and to place stent in such a manner that the SB wire is in the distal most strut of the part of stent strut covering the orifice of side branch, POT technique may be used to optimise size in the proximal part of stent.

6. The stent delivery system as claimed in claim 1 wherein Steps of rewiring the branches comprise: No rewiring is required whereas in existing or conventional technique following steps are required the MB wire (blue color) is now placed in side branch keeping SB wire as road map (FIG. 8 B), After placing MB wire into side branch; the SB wire is now taken out and placed in the main branch to ensure that now both the wires are inside the stent and further procedure on the side branch to make possible to perform without deforming the already deployed stent.

7. The stent delivery system as claimed in claim 1 wherein improved stent provided with channel/channels for SB wire between stent and underlying balloon with SB dilatation to be facilitated with change in strut design around the entry point of ‘channel’.

8. The stent delivery system as claimed in claim 7 wherein SB ostium coverage to be facilitated with change in strut design around the entry point of ‘channel’.

9. The stent delivery system as claimed in claim 1 wherein The strut through which the SB wire is passing (around the entry point of ‘channel’) formed in such a way that it accommodates the side branch (SB) size balloon.

10. The stent delivery system as claimed in claim 1 wherein the said stent made to minimise ‘sliding/folding door’ approach by minimizing heap of metal on wall opposite carina to decrease chance of SB restenosis as well as thrombosis.

11. The stent delivery system as claimed in claim 1 wherein a Slot for MB wire opposite to longer length of stent to guide proper position of stent in SB to facilitate for positioning of the stent at SB ostium.

12. The stent delivery system as claimed in claim 1 wherein the bevelled stent positioned in side branch to minimise stent protrusion in main branch.

13. The stent delivery system as claimed in claim 1 wherein a new stent design with longer Overhanging balloon’ in proximal part of stent to address issues of side branch ostium without protruding the stent into main branch.
